(19) United States
(12) Patent Application Publication
(10) Pub. No.: US 2011/0038916 A1
(43) Pub. Date: Feb. 17, 2011

(54) Sanitizers

(76) Inventor: Gilbert Buchalter, Millburn, NJ (US)

Correspondence Address: GILBERT BUCHALTER;PHARMACEUTICAL INNOVATIONS,, 897 FRELINGHUSEN AVENUE, NEWARK, NJ, 07114, US

(21) Appl. No.: 12/462,852

(22) Filed: Aug. 11, 2009

(51) Int. Cl. A61K 9/70; A61K 31/14; A61P 31/02

(52) U.S. Cl. 424/443; 514/643

(57) ABSTRACT

The present invention provides sanitizer systems for improving personal hygiene and reducing the potential for the spread of disease. The sanitizer systems enable more thorough cleansing by providing mechanically removal of microbial agents and soil from the skin with an absorbent sheet.

### FIELD OF THE INVENTION

The present invention provides sanitizer systems for improving personal hygiene and reducing the potential for the spread of disease. The sanitizer systems enable more thorough cleansing by providing mechanically removal of microbial agents and soil from the skin with an absorbent sheet.

### BACKGROUND OF THE INVENTION

Soap and water are efficient as a sanitizer because they mechanically remove microorganisms and soil, which are then washed away. Current hand sanitizers are used when soap and water are not available. Conventional hand sanitizers actually cause microorganisms and soil, alive or otherwise, to adhere to hands because they are formulated for air-drying without the use of a towel. Moreover, the alcohol used in many hand sanitizers evaporates too quickly to effectively sanitize the hands.

U.S. Pat. No. 7,426,994 (Simmons) discloses a personal hygiene article comprising an individually packaged sanitizing wipe, a second package, a graspable member and means for attaching the second package to a different article. The individually packaged sanitizing wipe includes a first package having a sanitizing wipe. The second package has the individually packaged sanitizing wipe. The graspable member is attached to a surface of the package in a manner enabling grasping and pulling of the graspable member to at least one of open the package and detach the package from the different article. The means for attaching the second package to a different article is attached to the second package.

U.S. Pat. No. 6,916,480 (Anderson et al.) discloses a wiper having a controlled release anti-microbial agent for providing anti-microbial cleansing of surfaces. The wiper is formed from an absorbent base web to which an anti-microbial formulation is adhered. The formulation includes an anti-microbial agent that is capable of being controllably released from the wiper. In some embodiments, a polymer mixture may be employed to control the rate of release of the anti-microbial agent. Various anti-microbial agents, such as metal ions and organic compounds may be employed.

U.S. Pat. No. 6,482,423 (Beerse et al. '423) and U.S. Pat. No. 6,197,315 (Beerse et al. '315) discloses an anti-microbial wipe comprising a porous or absorbent sheet impregnated with an anti-microbial cleansing composition. The anti-microbial cleansing composition comprises, by weight, from about 0.001% to about 5.0% of an anti-microbial active; from about 0.05% to about 10% of an anionic surfactant; from about 0.1% to about 10% of a proton donating agent; and from about 3% to about 99.85% of water. The composition is adjusted to a pH from about 3.0 to about 6.0. The anti-microbial cleansing composition has a Gram Positive Residual Effectiveness Index of greater than about 0.5 or 0.3, respectively, and a Mildness Index of less than about 0.3. The invention is said to encompass methods for cleansing skin and providing residual effectiveness versus Gram-positive bacteria using these products.

U.S. Pat. No. 5,888,524 (Cole) discloses a homogeneous anti-microbial composition and anti-microbial wet wipes and lotions, which include the anti-microbial composition. The homogeneous anti-microbial composition includes, by weight, at least about 50% water and an effective amount of a hydrophobic anti-microbial agent, which is uniformly dispersed in the composition. The anti-microbial wet wipe includes from about 150 to about 600% of the anti-microbial composition based on the dry weight of the wipe. The homogenous anti-microbial composition may include, by weight, (a) from about 0.01 to about 3% of a hydrophobic anti-microbial agent; (b) from about 1 to about 15% of an amide; (c) from about 1 to about 30% of a surfactant; and (d) from about 50 to about 98% of water. Cole at abstract. The hydrophobic anti-microbial agent is triclosan or triclocarban. The amide is selected from the group consisting of alkanolamides, long chain fatty acid diethanolamides, long chain fatty acid monoethanolamides, monoisopropanolamides, and combinations thereof.

U.S. Pat. No. 5,683,971 (Rose et al.) discloses an abrasive hand-cleaning article, which comprises a substrate having an abrasive surface and being capable of absorbing and retaining a fluid, and a nonabrasive aqueous hand cleanser absorbed in the substrate. The hand cleanser comprises one or both of a solvent and a surfactant whereby cleansing action is achieved by the solvent or surfactant, and abrasive cleansing action is achieved by the abrasive surface of the substrate. The substrate can comprise a cloth-like towel. A number of such towels are provided in a continuous rolled cylinder, which is housed in a sealable, airtight container. An opening in the lid of the container allows the user to remove individual towels. A waterless hand cleanser emulsion is absorbed on the substrate and comprises, by weight, 2-40% of a non-irritating emulsifiable organic solvent comprising an aliphatic liquid, a dibasic ester, vegetable oil, terpene, or a glycol ether; 2-20% of a surfactant; and 60-95% of water.

U.S. Pat. No. 4,620,502 (Kimble) discloses a device, which wets in a sanitizing solution and dispenses disposable paper towels. A continuous roll of dry paper towels is inserted into a canister within a larger container. The canister has an opening through which towels exit the canister into the interior of the larger container. External to the canister, but internal to the larger container, are broad, flat belts which engage the towels as they exit the canister and carry the towels through a sanitizing solution to an opening in the larger container. The belts are geared to and driven by a lever external to the larger container. When the lever is pulled, a sanitizing towelette is dispensed from the larger container.

United States patent application publication no. 2006/0003649 and PCT patent application WO 2006/025895 (Runge et al. '649) disclose a wet wipe product comprising a fibrous substrate material, a triggerable binder formulation, and a wetting composition comprising an insolubilizing agent in contact with the fibrous substrate material. The triggerable binder formulation is insoluble in the wetting composition and dispersible in disposal water and is selected from the group consisting of acrylamide polymers and polymer formulations, vinylamide/amine polymers and polymer formulations. The insolubilizing agent comprises a polyol.

United States patent application publication no. 2005/0106979 (Scheubel et al. '979) discloses a disposable flexible article useful for cleaning, sanitizing and disinfecting hard surfaces, adapted for inclusion of a cleaning solution in the core layer and for controlled and repeated release. The article comprises a first water-insoluble non-woven layer comprised of synthetic fibers having outer and inner surfaces, a water-insoluble core layer made of a cellulosic wadding material having a density less than 0.3 grams per cubic centimeter, having an stretch capacity of at least 30%, and having an absorbent capacity of at least 12 grams per gram of its basis weight; and a second water-insoluble non-woven layer comprised of synthetic fibers having outer and inner surfaces. The core layer is positioned between the inner surfaces of the first and second layers and the first, second and intermediate core layers are mechanically through-bonded by needling to form a unified article having perforations of less than 300 microns in diameter extending through all of the layers. The perforations are formed during the mechanical bonding. The perforations assist in controlling water absorption into the core for activating the cleaning solution and for the controlled and repeated release of the cleaning solution on its activation by the absorbed layer.

United States patent application publication no. 2003/0194932 and PCT patent application WO 2003/053144 (Clark et al. '932) disclose a wiper comprising a substrate and a sanitizing formulation applied to the substrate in an amount from about 150% to about 600% of the dry weight of the wiper. The sanitizing formulation comprises water and, by weight, from 0.01%-1% of an anti-microbial agent that includes a quaternary ammonium compound. The sanitizing formulation is capable of being released from the substrate as a solution such that the quaternary ammonium compound is present within the solution in an amount less than about 2000 parts per million of the solution, and the wiper exhibits a log reduction for E. Coli of at least about 2.

United States patent application publication no. 2003/0114069 (Scheubel et al. '069) discloses a disposable flexible article useful for cleaning, sanitizing, and disinfecting hard surfaces. The article comprises a first water-insoluble non-woven layer comprised of synthetic fibers having outer and inner surfaces, b) a water-insoluble non-woven cellulosic core layer, and c) a second water-insoluble non-woven layer having outer and inner surfaces. The core layer is positioned between the inner surfaces of the first and second layers and the first, second and intermediate core layer are mechanically through bonded by needling to form a unified article having perforations of less than 300 microns in diameter extending through all of the layers. The perforations are formed during the mechanical bonding.

PCT patent application WO 2006/124043 (Kelly) discloses a disposable flexible article useful for cleaning, sanitizing, and disinfecting hard surfaces, adapted for inclusion of a cleaning solution in the core layer and for controlled and repeated release. The article comprises (a) a first water-insoluble non-woven layer comprised of synthetic fibers having outer and inner surfaces, (b) a water-insoluble core layer made of a cellulosic material having a density less than 0.7 grams per cubic centimeter, having an stretch capacity of at least 30%, and having an absorbent capacity of at least 12 grams per gram of its basis weight, and (c) a second water-insoluble non-woven layer comprised of synthetic fibers having outer and inner surfaces. The core layer is positioned between the inner surfaces of the first and second layers and the first, second and intermediate core layers are ultrasonically through-bonded to form a unified article having perforations of less than 300 microns in diameter extending through all of the layers. The perforations are formed during the ultrasonic bonding, wherein the perforations assist in controlling water absorption into the core required for activating the cleaning solution and for the controlled and repeated release of the cleaning solution on its activation by the absorbed layer.

PCT patent application WO 2005/040328 (Peterson et al.) discloses pre-moistened, anti-microbial wipes that comprise a more abrasive side and a less abrasive side. The pre-moistened, anti-microbial wipes may comprise both cellulosic fibers and fibers selected from bi-component fibers, multi-component fibers, printed binder, and apertured film. The pre-moistened, anti-microbial wipes may be used as part of a cleaning implement.

PCT patent application WO 1993/07250 (Gluck) discloses an antiseptic cleansing composition comprising an alkanol and an aqueous concentrate of a surfactant. The compositions may also comprise a biocide. Gluck also discloses a method for cleansing skin comprising applying the composition onto skin allowing the alkanol on the skin to evaporate and then applying water to the dry skin and washing the skin with foam generation and finally rinsing the skin.

Therefore, there is a need to develop new sanitizers with antimicrobial agents that more efficiently lower the microbial count on skin and thereby reduce the potential for the spread of disease.

### SUMMARY OF THE INVENTION

The present invention provides a two-part sanitizer system comprising (a) a first part comprising a sanitizing composition having an antimicrobial agent; and (b) a second part comprising an absorbent sheet; wherein the first and second parts are separate.

The present invention also provides a method for sanitizing skin, which comprises (A) employing a two-part sanitizer system comprising: a) a first part comprising a sanitizing composition having an antimicrobial agent; and (b) a second part comprising an absorbent sheet; wherein the first and second parts are separate; (B) contacting the sanitizing composition with skin; and (C) contacting the absorbent sheet with the skin and sanitizing composition to mechanically remove microbial agents from the skin.

The present invention further provides a method for sanitizing skin, which comprises (A) employing a sanitizer system comprising an absorbent sheet with a sanitizing composition having an antimicrobial agent impregnated on the absorbent sheet; (B) contacting the absorbent sheet and sanitizing composition with skin; and (C) mechanically removing microbial agents from the skin with the absorbent sheet; wherein the sanitizing composition dries at a rate sufficiently slow such that the absorbent sheet is able to mechanically remove microbial agents from the skin.

The present invention still further provides a method for sanitizing skin, which comprises (A) employing a sanitizer system comprising an absorbent sheet with a sanitizing composition having an antimicrobial agent impregnated on the absorbent sheet; (B) to contacting the absorbent sheet and sanitizing composition with skin; and (C) mechanically removing microbial agents from the skin with the absorbent sheet; wherein the sanitizing composition will be transferred to the skin from the contacting and can be mechanically removed from the skin with the absorbent sheet before the absorbent sheet dries.

### DETAILED DESCRIPTION OF THE INVENTION

The present invention provides sanitizers that enable more efficient cleansing of microbial agents by providing mechanically removal of microbial agents and soil from the skin with an absorbent sheet.

The present invention provides a two-part sanitizer system comprising (a) a first part comprising a sanitizing composition having an antimicrobial agent; and (b) a second part comprising an absorbent sheet; wherein the first and second parts are separate.

The present invention also provides a method for sanitizing skin, which comprises (A) employing a two-part sanitizer system comprising: a) a first part comprising a sanitizing composition having an antimicrobial agent; and (b) a second part comprising an absorbent sheet; wherein the first and second parts are separate; (B) contacting the sanitizing composition with skin; and (C) contacting the absorbent sheet with the skin and sanitizing composition to mechanically remove microbial agents from the skin.

The present invention further provides a method for sanitizing skin, which comprises (A) employing a sanitizer system comprising an absorbent sheet with a sanitizing composition having an antimicrobial agent impregnated on the absorbent sheet; (B) contacting the absorbent sheet and sanitizing composition with skin; and (C) mechanically removing microbial agents from the skin with the absorbent sheet; wherein the sanitizing composition dries at a rate sufficiently slow such that the absorbent sheet is able to mechanically remove microbial agents from the skin.

The present invention still further provides a method for sanitizing skin, which comprises (A) employing a sanitizer system comprising an absorbent sheet with a sanitizing composition having an antimicrobial agent impregnated on the absorbent sheet; (B) contacting the absorbent sheet and sanitizing composition with skin; and (C) mechanically removing microbial agents from the skin with the absorbent sheet; wherein the sanitizing composition will be transferred to the skin from the contacting and can be mechanically removed from the skin with the absorbent sheet before the absorbent sheet dries.

As set out above, the sanitizer composition of the present invention comprises an antimicrobial agent. An antimicrobial agent is a substance that kills or inhibits the growth of microbial agents (microorganisms, microbes) such as bacteria, fungi, protozoans, and viruses. Antimicrobial agents either kill microbial agents (microbiocidal, biocide) or prevent the growth of microbial agents (microbiostatic, biostatic). Disinfectants are antimicrobial substances used on non-living objects. Antibiotic agents are generally used to treat bacterial infections. Antiviral agents are used specifically for treating viral infections. Most antiviral agents now available are designed to treat HIV, herpes viruses, the hepatitis B and C viruses, and influenza A and B viruses. Antifungal agents are used to treat fungal infections such as athlete's foot, ringworm, candidiasis (thrush), serious systemic infections such as cryptococcal meningitis, and others. Antiparasitics are used to treat parasites, such as nematodes, cestodes, trematodes, infectious protozoa, and amoebae. A wide range of chemical and natural agents including essential oils are also used as antimicrobial agents.

Antimicrobial agents are well known in the art and the exact choice of agent will depend upon the intended use of the sanitizer. Preferred antimicrobial agents in the present invention are methyl, ethyl, and isopropyl alcohols. More preferably, the antimicrobial agent is ethyl alcohol.

Other preferred antimicrobial agents are the quaternary ammonium compounds. A preferred quaternary ammonium compound is the commercially available benzalkonium chloride (alkyldimethylbenzylammonium chloride, ADBAC), which is a mixture of alkylbenzyldimethylammonium chlorides of various even-numbered alkyl chain lengths. This product is a nitrogenous cationic surface-acting agent belonging to the quaternary ammonium group. It has three main categories of use; as a biocide, a cationic surfactant and phase transfer agent.

The exact amount of antimicrobial agent to be used in the sanitizer composition will depend upon the particular agent utilized and the condition being treated. When the antimicrobial agent is a quaternary ammonium compound, the antimicrobial agent will be present in an amount from about 0.001% to about 5%, preferably from about 0.05% to about 1%, more preferably from about 0.05% to about 0.5% and more preferably from about 0.1% to about 0.25%, by weight of the antimicrobial agent in the sanitizing composition. When the antimicrobial agent is an alcohol, the antimicrobial agent will be present in an amount up to about 98%, preferably up to about 80%, more preferably from up to 70% and more preferably up to about 60%, by weight of the antimicrobial agent in the sanitizing composition.

The absorbent sheet (wipe, substrate, towelette, towel) in the present invention is a small piece of paper or cloth that often comes folded and individually wrapped for convenience. The absorbent sheet may be formed from any woven or non-woven fiber, cellulosic, or fiber mixture. Preferably, the towelette has a high absorbent capacity, from about 5 to about 20 grams/gram, more preferably from about 9 to about 20 grams/gram.

The absorbent capacity of a substrate is the ability of the substrate, while supported horizontally, to hold liquid. Methods for making woven and non-woven cloths are well know in the art.

The sanitizer system of the present invention may be used on skin to improve personal hygiene. Preferably, the sanitizer system is used on human skin, more preferably on human hands. The sanitizer system is designed to work in a two-step method. The first step is to contact the sanitizer composition containing the antimicrobial agent with skin for the purpose of cleaning the surface of the skin and controlling the growth and viability of microbial agents. In the second step, the absorbent sheet is used to mechanically remove the microbial agents and soil from the skin along with the sanitizer composition. The sanitizer system of the invention will decrease the microbial level on skin by a therapeutically effective amount. In general, the sanitizer system will decrease the microbial level on skin by at least 10%, preferably by at least 30%, more preferably by at least 50%, and most preferably by at least 90%.

The sanitizer compositions of the present invention may also include a wide range of optional ingredients such as cosmetic and pharmaceutical ingredients commonly used in the skin care industry. Non-limiting examples include abrasives, anti-caking agents, antioxidants, binders, bulking agents, chelating agents, chemical additives, colorants, astringents, denaturants, astringents, emulsifiers, analgesics, film formers, fragrance agents, opacifying agents, plasticizers, preservatives, skin-conditioning agents (emollients, humectants, miscellaneous), skin protectants, solvents, solubilizing agents, suspending agents, surfactants, and the like.

In the embodiments where the absorbent sheet is impregnated with a sanitizer composition, any method suitable for the application of the sanitizer composition can be used to impregnate the absorbent sheet. After coating (or non-coating), the sheets may be folded into stacks and packaged in any of the moisture and vapor-resistant packages known in the art. The sanitizer systems of the present invention are made via art recognized techniques for the various systems.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

1. A two-part sanitizer system comprising:
(a) a first part comprising a sanitizing composition having an antimicrobial agent; and
(b) a second part comprising an absorbent sheet;
wherein the first and second parts are separate.

2. The system according to claim 1, wherein the first and second parts are packages or dispensers.

3. The system according to claim 1, wherein both parts are absorbent sheets, the first part comprising the sanitizing composition impregnated on an absorbent sheet.

4. The system according to claim 1, wherein the antimicrobial agent is selected from the group consisting of methyl, ethyl, and isopropyl alcohols, and mixtures thereof, present in an amount up to about 98%, by weight.

5. The system according to claim 1, wherein the antimicrobial agent is a quaternary ammonium compound present in an amount from about 0.001% to about 5%, by weight.

6. The system according to claim 5, wherein the antimicrobial agent is benzalkonium chloride.

7. A method for sanitizing skin, which comprises:
(A) employing a two-part sanitizer system comprising:
(a) a first part comprising a sanitizing composition having an antimicrobial agent; and
(b) a second part comprising an absorbent sheet;
wherein the first and second parts are separate;
(B) contacting the sanitizing composition with skin; and
(C) contacting the absorbent sheet with the skin and sanitizing composition to mechanically remove microbial agents from the skin.

8. The system according to claim 7, wherein the first and second parts are packages or dispensers.

9. The system according to claim 7, wherein both parts are absorbent sheets, the first part comprising the sanitizing composition impregnated on an absorbent sheet.

10. The system according to claim 7, wherein the antimicrobial agent is selected from the group consisting of methyl, ethyl, and isopropyl alcohols, and mixtures thereof, present in an amount up to about 98%, by weight.

11. The system according to claim 7, wherein the antimicrobial agent is a quaternary ammonium compound present in an amount from about 0.001% to about 5%, by weight.

12. The system according to claim 12, wherein the antimicrobial agent is benzalkonium chloride.

13. A method for sanitizing skin, which comprises:
(A) employing a sanitizer system comprising an absorbent sheet with a sanitizing composition having an antimicrobial agent impregnated on the absorbent sheet;
(B) contacting the absorbent sheet and sanitizing composition with skin; and
(C) mechanically removing microbial agents from the skin with the absorbent sheet;
wherein the sanitizing composition dries at a rate sufficiently slow such that the absorbent sheet is able to mechanically remove microbial agents from the skin.

14. The system according to claim 13, wherein the antimicrobial agent is selected from the group consisting of methyl, ethyl, and isopropyl alcohols, and mixtures thereof, present in an amount up to about 98%, by weight.

15. The system according to claim 13, wherein the antimicrobial agent is a quaternary ammonium compound present in an amount from about 0.001% to about 5%, by weight.

16. The system according to claim 15, wherein the antimicrobial agent is benzalkonium chloride.

17. A method for sanitizing skin, which comprises:
(A) employing a sanitizer system comprising an absorbent sheet with a sanitizing composition having an antimicrobial agent impregnated on the absorbent sheet;
(B) contacting the absorbent sheet and sanitizing composition with skin; and
(C) mechanically removing microbial agents from the skin with the absorbent sheet;
wherein the sanitizing composition will be transferred to the skin from the contacting and can be mechanically removed from the skin with the absorbent sheet before the absorbent sheet dries.

18. The system according to claim 17, wherein the antimicrobial agent is selected from the group consisting of methyl, ethyl, and isopropyl alcohols, and mixtures thereof, present in an amount up to about 98%, by weight.

19. The system according to claim 18, wherein the antimicrobial agent is a quaternary ammonium compound present in an amount from about 0.001% to about 5%, by weight.

20. The system according to claim 19, wherein the antimicrobial agent is benzalkonium chloride.
